(19) United States
(12) Patent Application Publication
(10) Pub. No.: US 2006/0173290 A1
(43) Pub. Date: Aug. 3, 2006

(54) Localised medical instrument with tilt and swivel screen

(76) Inventors: Stephane Lavallee, Saint Martin D'Uriage (FR); Jocelyne Troccaz, Eybens (FR)

Correspondence Address: PLEVY & HOWARD, P.C., P.O. BOX 226, FORT WASHINGTON, PA, 19034, US

(21) Appl. No.: 10/548,556

(22) PCT Filed: Mar. 10, 2004
(86) PCT No.: PCT/FR04/50100

§ 371 Date: Jan. 30, 2006

(30) Foreign Application Priority Data
Mar. 10, 2003 (FR) 0302940

(51) Int. Cl. A61B 5/05

(52) U.S. Cl. 600/424

(57) ABSTRACT

The invention relates to a medical instrument comprising a medical tool intended, while a medical act is being performed, to be maintained by a user and/or by a robot and the position of which is intended to be determined by a tracking system, and comprising a display system having a visual control screen, a connection element connecting the display system to the tool and maintaining the minimum distance between the visual control screen and the tool under 15 centimeters and being capable of orienting the visual control screen with respect to the tool, means for transmitting to the display system data to be displayed on the visual control screen which depend on the determined position of the tool, means for supplying the display system, and a sterile enclosure containing the visual control screen, the transmission means, and the supply means.

The present invention relates to medical instruments used to perform computer-assisted medical acts.

When a computer-assisted surgical act is performed, for example, a surgical operation, a therapeutic act or a diagnosis, the position of the medical instrument used by the doctor is generally determined in real time with respect to an absolute reference frame. From the position thus determined, a processing unit provides “useful” data to the doctor via a man/machine interface.

When the medical instrument is maintained directly by the doctor, its position is generally determined by an optical, magnetic, etc. localization system. When the instrument is maintained by a carrying robot assisting the doctor, its position is generally directly determined from a performance model of the carrying robot. Generally, the processing unit is a computer and the man/machine interface corresponds to the computer display screen. According to the nature of the medical instrument and of the medical act to be performed, the useful data for example correspond to the optimal trajectory to be followed by the instrument.

A disadvantage of the use of a computer display screen for the display of useful data is that the doctor must look away from the medical instrument to consult the data displayed on the screen. Such repeated motions risk disturbing the doctor's concentration. The display screen may be maintained at the end of a carrier arm close to the instrument to ease the data reading rapidity. However, the display screen cannot be brought too close to the medical instrument to avoid hindering the doctor's motions and field of vision. The doctor must thus always look away from the instrument to consult the useful displayed data.

There exist display systems comprising a transparent screen placed between a user and a tool handled by the user. Useful data are displayed directly on the transparent screen. The user can thus, without looking away, monitor the tool through the transparent screen or consult the data displayed on the screen. The transparent screen is for example formed of a transparent visor associated with a helmet worn by the doctor.

Such “transparency” display systems are however complex and of high cost. Further, they are poorly adapted to the performing of medical acts. Indeed, such systems being necessarily placed close to the patient, and possibly directly in contact with the doctor, they must comply with sterilization constraints specific to the medical field. However, current transparency display systems are poorly adapted to conventional sterilization techniques.

The present invention provides a medical instrument, for performing a computer-assisted medical act, the position of which is determined for the display of data that can be consulted by the doctor performing the medical act without requiring for the latter to excessively look away from the medical instrument.

For this purpose, it provides a medical instrument comprising a medical tool capable, while a medical act is being performed, of being maintained by a user and/or by a robot and the position of which can be determined by a tracking system. The medical instrument comprises a display system having a visual control screen, a connection element connecting the display system to the tool and maintaining the minimum distance between the visual control screen and the tool under 15 centimeters and being capable of orienting the visual control screen with respect to the tool, means for transmitting to the display system data to be displayed on the visual control screen which depend on the determined position of the tool, means for supplying the display system, and a sterile enclosure containing the visual control screen, the transmission means, and the supply means.

According to an embodiment of the invention, the connection element is sterilizable.

According to an embodiment of the invention, the enclosure comprises a sterile, rigid, and transparent package containing the display system.

According to an embodiment of the invention, the supply means and the transmission means comprise a supply cable connected to the display system, the enclosure comprising a sterile sheath at least partially surrounding the supply cable.

According to an embodiment of the invention, the enclosure comprises a sterile, flexible, and transparent protective cover containing the display system.

According to an embodiment of the invention, the display system is sterile.

According to an embodiment of the invention, the connection element is capable of orienting the visual control screen with respect to the tool according to discrete positions.

According to an embodiment of the invention, the visual control screen is a touch-sensitive screen.

According to an embodiment of the invention, the connection element is capable of being separated from the tool and/or from the display system.

According to an embodiment of the invention, the transmission means are remote data transmission means integrated to the display system.

According to an embodiment of the invention, the supply means are a battery integrated to the display system.

The foregoing object, features, and advantages of the present invention, as well as others, will be discussed in detail in the following non-limiting description of specific embodiments in connection with the accompanying drawings, among which:

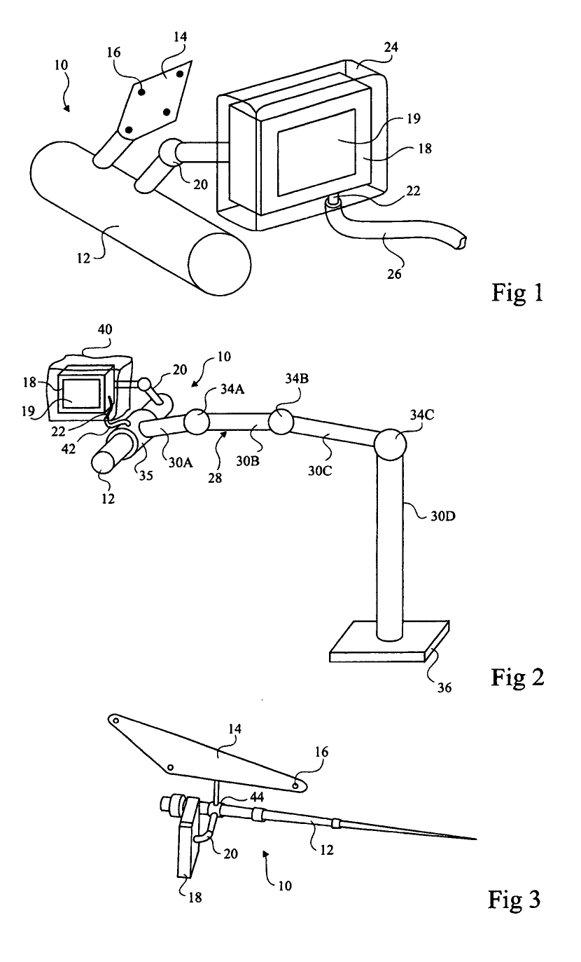

FIGS. 1, 2, and 3 show simplified perspective views of first, second, and third examples of embodiment of an instrument according to the invention.

FIG. 1 shows a first example of embodiment of a medical instrument 10 according to the invention used to perform a computer-assisted medical act. Instrument 10 comprises a conventional medical tool 12 schematically shown as a cylindrical body.

Example of medical tools conventionally used to perform computer-assisted medical acts are the following:

bone cutting guide or bone cutting guide blocks;

oscillating saw;

motor comprising a drill, of drilling machine type, for example for the spine or to perform operations such as femur neck sights, or sacroiliac sights, guide of such a motor;

milling cutter, milling cutter guide;

needle, for example, puncture needle or grain placing needle in curietherapy, needle guide;

biopsy trocar, trocar guide;

electrode, for example, stimulation or measurement electrode, electrode guide;

pointer, pointer guide;

flexible or rigid endoscope; and

ultrasound scanning probe.

According to the first example of embodiment, tool 12 is to be maintained by the doctor while the medical act is being performed. Medical instrument 10 also comprises tracking means 14 attached to tool 12 and for example formed of a rigid body on which are arranged back-reflective disks 16. Rigid body 14 is used for the determination of the position of instrument 10 by an optical-type tracking instrument not shown.

A display system 18 comprising a visual control screen 19 is connected to cylindrical body 12 by an articulated mechanical joint element 20, formed, for example, by two arms connected by a ball. Display system 18 is of small dimensions, visual control screen 19 being preferably as large as possible. Display system 18 is for example comprised in a cube having a side smaller than 20 cm. Connection element 20 is capable of maintaining the minimum distance between visual control screen 19 and the tool smaller than 15 centimeters. Mechanical joint element 20 enables the doctor to change the orientation of display system 18 with respect to tool 12 continuously or according to predefined discrete orientation positions. A supply cable 22 is connected to display system 18 and transmits the supply power and the useful data to be displayed on visual control screen 19 thereto.

The nature of the useful data depends on the type of used instrument and on the medical act to be performed. Certain useful data depend on the position of medical instrument 10 and vary in real time according to the position of medical instrument 10. The data displayed on visual control screen 19 may be images, graphics, text, etc., that is, any data capable of bringing to the doctor assistance or pertinent information during the performing of the medical act.

Examples of useful data displayed on visual control screen 19 are the following:

sighting device materializing the ideal trajectory that medical instrument 10 must follow, the trajectory really followed by instrument 10 being also shown. Such data especially eases surgical acts comprising linear motions such as a drilling, a puncture, a biopsy, a placing of electrodes or needles;

ideal cutting plane with respect to a really followed cutting plane;

cross-section view of different organs provided by anatomic or functional imaging means perpendicular to the trajectory of instrument 10;

models of organs defined beforehand and shown on visual control screen 19 according to the position of instrument 10;

parameters adapted to a given medical act, for example, anteversion and inclination angles of organs such as for example in the case of knee and/or hip arthroplasty, anisometry maps such as for example in the case of the reconstruction of the anterior cruciate ligament, or depth reached for a linear sighting gesture;

image acquired by instrument 10 in the case where instrument 10 is an ultrasound scanning probe or an endoscopic probe, possibly with the position of a target provided beforehand.

The data displayed on visual control screen 19 may be a combination of several data, especially from among the previously-mentioned examples.

Display system 18 is contained in a transparent package 24 which does not impair the vision of visual control screen 19 by the doctor. The portion of supply cable 22 of display system 18 outside of transparent package 24 is contained in a sheath 26. Sheath 26, transparent package 24, and mechanical connection element 20 are made of materials that can easily be sterilized. A sterilization is for example performed between two successive uses. Transparent package 24, sheath 26, and connection element 20 may also be replaced with new sterile elements if their selfcost enables it.

Connection element 20 is dismountable and can be detached from tool 12 and from display system 18. Transparent package 24, sheath 26, and connection element 20 can thus be sterilized separately for a next use. Further, a same display system 18 can also be attached to different tools 12.

Medical instrument 10 according to the first example of ebodiment of the invention is advantageously capable of being maintained directly by a doctor. For this purpose, the weight of display system 18 is as small as possible to avoid excessively tiring the doctor.

According to a variation of the first example of embodiment, package 24 comprises a transparent area only at the level of visual control screen 19.

Mechanical connection element 20 is such that the average distance between visual control screen 19 and tool 12 remains smaller than 50 centimeters. Visual control screen 19 remains close to tool 12 while the medical act is being performed and is thus permanently in the doctor's field of vision. The doctor can thus consult the data displayed on visual control screen 19 and look away as little as possible.

FIG. 2 shows a second example of embodiment of a medical instrument 10 according to the present invention comprising, as for the first embodiment, a conventional tool 12 connected to a display system 18 having a visual control screen 19 by an adjustable mechanical joint element 20.

In the second example of embodiment, instrument 10 is maintained during the medical act by a carrying robot 28. The displacements of carrying robot 28 are remotely controlled by the doctor or automatically accompany the motions of instrument 10 manually guided by the doctor. As an example, carrying robot 28 is schematically represented by first, second, and third arms 30A, 30B, 30C assembled in series and connected by pivoting connections 34A, 34B, 34C. First arm 30A comprises a gripper 35 maintaining tool 12. Third arm 30D rests on the ground via a base 36. Electric motors, not shown, ensure the motions of the arms with respect to one another.

According to the second example of embodiment, display system 18 is contained in a flexible transparent protective cover 40 formed of a material that can easily be sterilized. The supply of display system 18 and the transmission of the data to be displayed on visual control screen 19 are provided by supply cable 22. The portion of supply cable 22 outside of sterile protective cover 40 is contained in a flexible sterile socket 42. Supply cable 22 may directly be connected to carrying robot 28 which ensures the transmission of the supply power and of the data to be displayed. The control signals of the electric motors of carrying robot 28 and the data to be displayed on visual control screen 19 are provided to carrying robot 28 by means not show. As for the first example of embodiment, mechanical joint element 20 is dismountable.

The second example of embodiment enables use of a display system 18 heavier than in the first example of embodiment since the weight of instrument 10 is supported by carrying robot 28. A display system integrating wireless data exchange means may further be provided to suppress supply cable 22. However, the dimensions of display system 18 must remain small enough not to disturb the doctor as he performs the medical act.

Further, the use of a transparent protective cover 40 enables the doctor performing the medical act to touch visual control screen 19. According to a variation of the second example of embodiment, the visual control screen is a touch-sensitive screen comprising embarked processing means. The doctor then selects by means of different menus the data that he desires to consult among the available data.

The use of a carrying robot 28 also enables determining the position of medical instrument 10 directly from a model representing carrying robot 28. This enables avoiding addition of a rigid body on tool 12 and thus reduces the bulk of instrument 10.

FIG. 3 shows a third example of embodiment of a medical instrument 10 according to the invention. Medical tool 12 shown in FIG. 3 corresponds, for example, to a mechanical puncture needle guide. Medical tool 12 is maintained in a reception ring 44 to which are attached rigid body 14 and mechanical joint element 20. Rigid body 14 has, in this example, a triangular shape, a disk 16 being arranged in each corner of rigid body 14. Mechanical connection element 20 is formed, for example, of an articulated elbow enabling orientation of display system 18.

According to the third example of embodiment, display system 18 is directly sterilized. Visual control screen 19 and its support then form a sterile enclosure. The transparent package of the first example of embodiment and the transparent protective cover of the second example of embodiment are then suppressed. The sterilization is for example performed in known manner by an exposure of display system 18 to gamma rays. Display system 18 may be used to perform several medical acts by being sterilized between each act. If the selfcost is sufficiently low, sterile display system 18 is disposable.

The supply of display system 18 is obtained by batteries directly integrated to display system 18. Further, display system 18 comprises means of remote data exchange for receiving the useful data to be displayed on the visual control screen. It is then not necessary to provide a supply cable and an associated sterile sheath as for the preceding embodiments.

According to a variation of the examples of embodiments, a separate control system is provided to modify the display of useful data on the visual control screen. It is for example a pedal control system, or a vocal control system actuated by the doctor during the operation.

The present invention has many advantages:

First, by providing a visual control screen directly at the level of the medical instrument used to perform a computer-assisted medical act, the present invention enables the doctor to consult useful data displayed on the visual control screen without looking away from the medical instrument.

Second, the present invention disturbs as little as possible the doctor's field of vision and motions as he performs the medical act, the display system being of reduced dimensions and being adjustable with respect to the tool forming the medical instrument.

Third, the present invention is particularly well adapted to the medical field since the elements forming the medical instrument can easily be sterilized. Further, said elements being dismountable, a separate sterilization of each element can be provided according to a sterilization technique adapted to each of them.

Of course, the present invention is likely to have various alterations and modifications which will readily occur to those skilled in the art. In particular, the characteristics of the third example of embodiment may be adapted to the first or to the second examples of embodiment. For example, in the first example of embodiment, display system 18 may integrate a supply battery and, possibly, remote data exchange means. This enables suppressing supply cable 22 and thus simplifying the structure of transparent package 24 as well as the retrieval and the assembly of transparent package 24 between two successive uses. The same holds true for the second embodiment.

1.-11. (canceled)

12. A medical equipment including:
a medical instrument (10) comprising a medical tool (12) capable, while a medical act is being performed, of being maintained by a user and/or by a robot (28), a display system (18) having a visual control screen (19), a connection element (20) connecting the display system to the tool and maintaining the minimum distance between the visual control screen and the tool under 15 centimeters and being capable of continuously or discretely orienting the visual control screen with respect to the tool and a sterile enclosure (24, 26, 40, 42) containing the visual control screen;
a tracking system (14, 16) for determining the position of the tool;
means (22) for transmitting to the display system data to be displayed on the visual control screen which depend on the determined position of the tool; and
means (22) for supplying the display system, the sterile enclosure containing the transmission means and the supply means.

13. The equipment (10) of claim 12, wherein the connection element (20) is sterilizable.

14. The equipment (10) of claim 12, wherein the enclosure comprises a sterile, rigid, and transparent package (24) containing the display system (18).

15. The equipment (10) of claim 12, wherein the supply means and the transmission means comprise a supply cable (22) connected to the display system (18), the enclosure comprising a sterile sheath (26) at least partially surrounding the supply cable.

16. The equipment (10) of claim 12, wherein the enclosure comprises a sterile, flexible, and transparent protective cover (40) containing the display system (18).

17. The equipment (10) of claim 12, wherein the display system (18) is sterile.

18. The equipment (10) of claim 12, wherein the visual control screen (19) is a touch-sensitive screen.

19. The equipment (10) of claim 12, wherein the connection element (20) is capable of being separated from the tool (12) and/or from the display system (18).

20. The equipment (10) of claim 12, wherein the transmission means are remote data transmission means integrated to the display system (18).

21. The equipment (10) of claim 12, wherein the supply means are a battery integrated to the display system (18).
